(19) United States
(12) Patent Application Publication
(10) Pub. No.: US 2021/0153846 A1
(43) Pub. Date: May 27, 2021

(54) METHODS AND APPARATUSES FOR PULSED WAVE DOPPLER ULTRASOUND IMAGING

(71) Applicants: Kirthi Bellamkonda, New Haven, CT (US); David Elgena, Jersey City, NJ (US); Cristina Shin, San Francisco, CA (US); Karl Thiele, Andover, MA (US); Christophe Meyer, New York, NY (US); Brandon Lane, Atlanta, GA (US)

(72) Inventors: Kirthi Bellamkonda, New Haven, CT (US); David Elgena, Jersey City, NJ (US); Cristina Shin, San Francisco, CA (US); Karl Thiele, Andover, MA (US); Christophe Meyer, New York, NY (US); Brandon Lane, Atlanta, GA (US)

(73) Assignee: Butterfly Network, Inc., Guilford, CT (US)

(21) Appl. No.: 17/103,059

(22) Filed: Nov. 24, 2020

Related U.S. Application Data
(60) Provisional application No. 62/940,367, filed on Nov. 26, 2019.

(51) Int. Cl. A61B 8/08; A61B 8/00; A61B 8/06

(52) U.S. Cl. CPC A61B 8/488; A61B 8/5207; A61B 8/461; A61B 8/06; A61B 8/0883; A61B 8/0891; A61B 8/4494

(57) ABSTRACT

Aspects of the technology described herein relate to automatically calculating an ultrasound pulse transmission direction and configuring an ultrasound device to transmit ultrasound pulses along the ultrasound pulse transmission direction for pulsed wave Doppler ultrasound imaging. Automatically calculating the ultrasound pulse transmission direction may be based on a selected sample volume within a subject where flow velocity is to be measured with the pulsed wave Doppler ultrasound imaging, and a selected direction of the flow velocity to be measured with the pulsed wave Doppler ultrasound imaging.

### CROSS-REFERENCE TO RELATED APPLICATIONS

The present application claims the benefit under 35 U.S.C. § 119(e) of U.S. Patent Application Ser. No. 62/940,367, filed Nov. 26, 2019 under Attorney Docket No. B 1348.70170US00, and entitled “METHODS AND APPARATUSES FOR PULSED WAVE DOPPLER ULTRASOUND IMAGING,” which is hereby incorporated by reference herein in its entirety.

### FIELD

Generally, the aspects of the technology described herein relate to imaging. Some aspects relate to pulsed wave Doppler ultrasound imaging.

### BACKGROUND

Ultrasound probes may be used to perform diagnostic imaging and/or treatment, using sound waves with frequencies that are higher than those audible to humans. Ultrasound imaging may be used to see internal soft tissue body structures. When pulses of ultrasound are transmitted into tissue, sound waves of different amplitudes may be reflected back towards the probe at different tissue interfaces. These reflected sound waves may then be recorded and displayed as an image to the operator. The strength (amplitude) of the sound signal and the time it takes for the wave to travel through the body may provide information used to produce the ultrasound image. Many different types of images can be formed using ultrasound devices. For example, images can be generated that show two-dimensional cross-sections of tissue, blood flow, motion of tissue over time, the location of blood, the presence of specific molecules, the stiffness of tissue, or the anatomy of a three-dimensional region.

### SUMMARY

According to one aspect of the application, an apparatus includes a processing device in operative communication with an ultrasound device, the processing device configured to automatically calculate an ultrasound pulse transmission direction for pulsed wave Doppler ultrasound imaging.

In some embodiment, the processing device is further configured to configure the ultrasound device to transmit ultrasound pulses along the ultrasound pulse transmission direction.

In some embodiment, the processing device is configured to automatically calculate the ultrasound pulse transmission direction based on a selected sample volume within a subject where flow velocity is to be measured with the pulsed wave Doppler ultrasound imaging and a selected direction of the flow velocity to be measured with the pulsed wave Doppler ultrasound imaging. In some embodiment, the processing device is configured, when automatically calculating the ultrasound pulse transmission direction for the pulsed wave Doppler ultrasound imaging, to: receive a selection of a sample volume within a subject where flow velocity is to be measured with the pulsed wave Doppler ultrasound imaging, and a selection of a direction of the flow velocity to be measured with the pulsed wave Doppler ultrasound imaging; and iterate through multiple ultrasound pulse transmission directions to determine a selected ultrasound pulse transmission direction for which an angle between the ultrasound pulse transmission direction and the direction of the flow velocity is closest to a particular optimal correction angle.

In some embodiment, the processing device is further configured to display an indication of the selected ultrasound pulse transmission direction and an indication of the angle between the ultrasound pulse transmission direction and the direction of the flow velocity. In some embodiment, the processing device is configured, when displaying the indication of the selected ultrasound pulse transmission direction, to: display, on a graphical user interface that depicts an ultrasound image collected by the ultrasound device, a line extending from a top of the ultrasound image, through an indication of the sample volume, and to a bottom of the ultrasound image, such that a path of the line in the ultrasound image corresponds to the selected ultrasound pulse transmission direction.

In some embodiment, the processing device is configured, when iterating through the multiple ultrasound pulse transmission directions, to iterate through multiple starting positions for transmission of the ultrasound pulses from the ultrasound device. In some embodiment, the processing device is configured, when iterating through the multiple starting positions for transmission of the ultrasound pulses from the ultrasound device, to iterate through multiple subsets of ultrasound transducers in a transducer array of the ultrasound device from which to transmit the ultrasound pulses. In some embodiment, the processing device is configured, when iterating through the multiple starting positions for transmission of the ultrasound pulses from the ultrasound device, to: for each respective starting position of the multiple starting positions for transmission of the ultrasound pulses from the ultrasound device, determine an angle that would be between the ultrasound pulses directed from the respective starting position to the sample volume and the direction of the flow velocity to be measured; and select a starting position for transmission of the ultrasound pulses such that the angle is closest to the particular optimal correction angle.

In some embodiment, the particular optimal correction angle is zero. In some embodiment, the processing device is configured to use a zero angle for the particular optimal correction angle for cardiac imaging. In some embodiment, the processing device is configured to use a nonzero angle for the particular optimal correction angle for carotid and/or vascular access imaging. In some embodiment, the processing device is configured to use a zero angle for the particular optimal correction angle for cardiac imaging and a nonzero angle for the particular optimal correction angle for carotid and/or vascular access imaging. In some embodiment, the processing device is configured to provide a user with an option to input the particular optimal correction angle.

In some embodiment, the processing device is configured, when automatically calculating the ultrasound pulse transmission direction, to determine whether the calculated ultrasound pulse transmission direction is larger than a particular limit angle. In some embodiment, the processing device is configured, if the calculated ultrasound pulse transmission direction is larger than the particular limit angle, to select a second ultrasound pulse transmission direction. In some embodiment, an angle between the second ultrasound pulse transmission direction and the direction of the flow velocity is next closest to the particular optimal correction angle after the ultrasound pulse transmission direction determined to be larger than the particular limit angle. In some embodiment, the processing device is configured to use a first angle for the particular limit angle for carotid and/or vascular access imaging that is smaller than a second angle used for the limit angle for cardiac imaging.

Some aspects include a method for using a processing device to perform the functions described above. Some aspects include at least one non-transitory computer-readable storage medium storing processor-executable instructions that, when executed by at least one processor on a processing device, cause the at least one processor to perform the functions described above.

### BRIEF DESCRIPTION OF THE DRAWINGS

Various aspects and embodiments of the application will be described with reference to the following figures. It should be appreciated that the figures are not necessarily drawn to scale. Items appearing in multiple figures are indicated by the same reference number in all the figures in which they appear.

FIG. 1 illustrates an example graphical user interface (GUI) for pulsed wave Doppler ultrasound imaging, in accordance with certain embodiments described herein;

FIG. 2 illustrates another example of the GUI of FIG. 1 for pulsed wave Doppler ultrasound imaging, in accordance with certain embodiments described herein;

FIG. 3 illustrates a flow chart of an example process for pulsed wave Doppler ultrasound imaging, in accordance with certain embodiments described herein; and

FIG. 4 illustrates a schematic block diagram of an example ultrasound system 400 upon which various aspects of the technology described herein may be practiced.

### DETAILED DESCRIPTION

Blood brings oxygen to tissues, and oxygen is important for tissues to survive. In light of this fact, measuring blood flow through a number of major blood vessels is an important diagnostic tool. When blood vessels are narrow, the usual amount of blood has to go through a smaller opening, so the velocity of the blood has to speed up. While color Doppler ultrasound imaging may indicate qualitatively whether flow is present or not, and may be useful in emergencies or for line placement, it may only be able to indicate whether there is some flow or zero flow. Having “some flow” may range from perfectly normal to “on the brink of loss of flow.” In pulsed wave Doppler ultrasound imaging, the shape of a pulsed wave output waveform, the change in shape along a blood vessel, the speed, and the ratio of speed from one point to another may all be used to determine whether a blood vessel is open (patent) or narrow (stenosed), and may help to catch problems with the blood vessels before a critical emergency happens.

One clinical use case for pulsed wave Doppler ultrasound imaging is carotid artery stenosis (CAS) screening. The prevalence of CAS in elderly men reaches 12.5% and can increase the risk for stroke. CAS is usually asymptomatic, and screening is traditionally performed after someone has a sentinel event like a transient ischemic attack (TIA), which is a mini-stroke that resolves within 24 hours completely. If screening were easier, then more people may be preemptively treated, especially since treatment is growing safer every single year and there have been large advancements in safe and minimally invasive treatment. Other anatomical regions where flow may be monitored with pulsed wave Doppler ultrasound imaging in the context of vascular surgery include the legs, the aorta, iliac vessels, subclavian vessels, and vertebral arteries. Additionally, pulsed wave Doppler ultrasound imaging may be used to monitor the flow through fistulas.

In obstetrics, high risk pregnancies usually fall into two categories: sufficiently high risk as to indicate immediate delivery, or low enough risk that monitoring on a twice-monthly basis is acceptable. In between those two categories may be a small slice of cases where changes on a 48-hourly basis may be an indication for delivery, because of rapid and acute changes in how much blood the fetus receives. This blood flow may be measured using pulsed wave Doppler ultrasound imaging of the umbilical artery. If the flow in the artery goes backwards when the heart muscle is relaxed, this may be very bad for the fetus. There are women who may require pulsed wave Doppler ultrasound imaging 3 to 4 times a week to check this. Additional applications of pulsed wave Doppler ultrasound imaging may include penile flow and testicular torsion.

Conventional ultrasound systems for performing ultrasound imaging like pulsed wave Doppler ultrasound imaging are large, complex, and expensive systems that are typically only purchased by large medical facilities with significant financial resources. Recently, less expensive and less complex ultrasound imaging devices have been introduced. Such imaging devices may include ultrasonic transducers monolithically integrated onto a single semiconductor die to form a monolithic ultrasound device. Aspects of such ultrasound-on-a chip devices are described in U.S. patent application Ser. No. 15/415,434 titled “UNIVERSAL ULTRASOUND DEVICE AND RELATED APPARATUS AND METHODS,” filed on Jan. 25, 2017 (and assigned to the assignee of the instant application) and published as U.S. Pat. Pub. No. US-2017-0360397-A1, which is incorporated by reference herein in its entirety. Such an ultrasound device may be in operative communication with a processing device, such as a smartphone or a tablet, that has a touch-sensitive display screen. The processing device may display ultrasound images generated from ultrasound data collected by the ultrasound device.

The inventors have developed technology for assisting a user in selecting parameters for pulsed wave Doppler ultrasound imaging using a processing device with a touch screen. The technology includes graphical user interfaces for selecting the location of a sample volume and the direction of the flow velocity to be measured. Furthermore, the inventors have developed technology for automatically determining an optimal ultrasound pulse transmission direction based on the sample volume and the direction of the flow velocity to be measured. Rather than requiring the user to manually select the ultrasound pulse transmission direction, this may enable easier and more efficient selection of parameters for pulsed wave Doppler ultrasound imaging.

It should be appreciated that the embodiments described herein may be implemented in any of numerous ways. Examples of specific implementations are provided below for illustrative purposes only. It should be appreciated that these embodiments and the features/capabilities provided may be used individually, all together, or in any combination of two or more, as aspects of the technology described herein are not limited in this respect.

In pulsed wave Doppler ultrasound imaging, ultrasound pulses are directed at a particular portion of a subject in which something (e.g., blood) is flowing. This allows for measurement of the velocity of the flow. Generally, the parameters for pulsed wave Doppler ultrasound imaging include:

1. The portion of the subject where the flow velocity is to be measured, which may also be referred to as the sample volume;

2. The direction of the flow velocity to be measured. In other words, if flow occurs in an arbitrary direction, the component of the velocity of that flow along this particular selected direction may be the velocity measured; and

3. The direction in which the ultrasound pulses are transmitted from the ultrasound device, and in particular, from the transducer array of the ultrasound device, to the sample volume.

In some embodiments, in an ideal scenario, the ultrasound pulses would travel through the sample volume in a direction exactly opposite the direction of the flow velocity to be measured. However, the direction of the ultrasound pulses may be constrained by the geometry of the transducer array of the ultrasound device and its position relative to the sample volume. If the direction of transmission of the ultrasound device is not the same as the direction of the flow velocity to be measured, there may be a correction angle, namely the angle between the direction of transmission and the direction of flow velocity. When measuring velocity at the sample volume, the processing device may perform a correction based on the correction angle, and this correction may reduce the resolution of the velocity measurement. Thus, in some embodiments, a correction angle as close as possible to 0 degrees may be preferable. A correction angle as close as possible to 0 degrees may also be preferable because, for a given sample volume size, larger correction angles may result in measuring multiple different flow velocities, because flow velocity may decrease towards the wall of a vessel in which the flow (e.g., blood flow) is occurring. A correction angle as close as possible to 0 may also result in a sharper velocity trace and less spectral spread.

In some embodiments, a non-zero correction angle may be preferable. For example, a non-zero correction angle may increase the measurable velocity scale range. Additionally, a non-zero correction angle may enable the ultrasound pulse to travel to the sample volume with less attenuation, reflection, and/or distortion (depending on the anatomy regions or structures through which the ultrasound pulse travels). For example, a vessel wall may reflect the ultrasound pulse more if the incident angle is close to 0 (aligned along the vessel wall) than if the incident angle to the vessel wall is more perpendicular (in a manner similar to the concept of “total internal reflection” in optics). The particular angle may be any angle greater than 0 degrees but less than or equal to 90 degrees. Non-limiting examples include 5, 10, 15, 20, 25, 30, 35, 40, 45, 50, 55, 60, 65, 70, 75, 80, 85, and 90 degrees. In some embodiments, for cardiac imaging, a zero correction angle may be preferable, while for carotid and vascular access imaging, a non-zero correction angle may be preferable.

As will be described below, the processing device may determine a “best” transmission direction based on the location of the sample volume and the direction of the flow velocity to be measured. In other words, a user may select the sample volume and the direction of flow velocity, and based on these selections, the processing device may determine the transmission direction. In some embodiments, the processing device may determine the transmission direction based on a predetermined optimal correction angle (e.g., 0 degrees or a non-zero correction angle). In some embodiments, a user may input the optimal correction angle, and the processing device may determine the transmission direction based on the user-inputted optimal correction angle. The following description describes how a user may select the sample volume and direction of flow velocity using a graphical user interface (GUI).

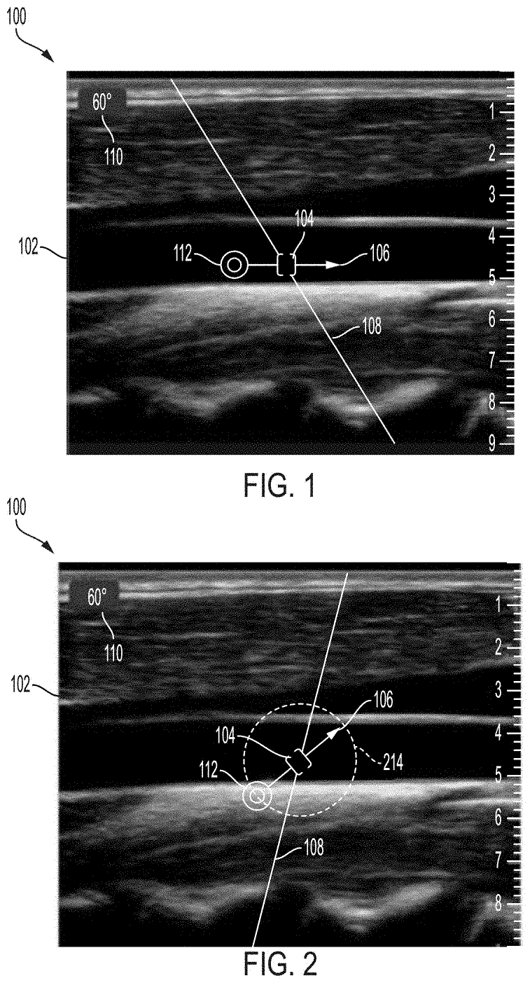

FIG. 1 illustrates an example GUI 100 for pulsed wave Doppler ultrasound imaging, in accordance with certain embodiments described herein. The GUI 100 may be displayed by a processing device in communication with an ultrasound device. For example, the processing device may be a mobile phone, tablet, or laptop. The ultrasound device and the processing device may communicate over a wired communication link (e.g., over Ethernet, a Universal Serial Bus (USB) cable or a Lightning cable) or over a wireless communication link (e.g., over a BLUETOOTH, WiFi, or ZIGBEE wireless communication link). The GUI 100 includes an ultrasound image 102, a sample volume 104, a vessel line 106, a beam line 108, an angle indicator 110, and a caliper 112.

The ultrasound image 102 may be the most recently collected ultrasound image by the ultrasound device. Thus, as the ultrasound device collects each ultrasound image (or ultrasound data for generating an ultrasound image), the ultrasound device may transmit the image or data to the processing device for updating the ultrasound image 102 on the GUI 100.

The location of the sample volume 104 in the ultrasound image 12 may determine the sample volume within the subject. In particular, every location in the ultrasound image 102 may correspond to a location within the subject. The processing device may configure the ultrasound device to specifically collect ultrasound data for measuring velocity from the location within the subject corresponding to the location of the sample volume 104 in the ultrasound image 102. The vessel line 106 (which extends from the caliper 112, through the sample volume 104, and to a terminal arrow) may determine the direction (as indicated by the arrow of the vessel line 106) of the flow for which the velocity is to be measured. As described above, this direction may determine, at least in part, the direction of the ultrasound pulse transmission and the correction angle. The beam line 108 extends from the top of the ultrasound image 102, through the sample volume 104, and to the bottom of the ultrasound image 102. The beam line 108 may indicate the direction in which the ultrasound device transmits ultrasound pulses. In particular, the processing device may configure the ultrasound device to transmit ultrasound pulses along a direction in the subject corresponding to the direction of the beam line 108 in the ultrasound image 102. The ultrasound transmission occurs from the ultrasound device to the subject, which may correspond to a direction along the beam line 108 from the top of the ultrasound image 102 to the bottom of the ultrasound image 102. The angle indicator 110 may indicate the correction angle, namely the angle between the beam line 108 and the vessel line 106.

In some embodiments, the caliper 112 may enable modification of the vessel line 106. In some embodiments, upon receiving a selection of the caliper 112 (e.g., by a user touching or clicking on the caliper 112), the processing device may display the GUI 100 of FIG. 2. FIG. 2 illustrates another example of the graphical user interface (GUI) 100 for pulsed wave Doppler ultrasound imaging, in accordance with certain embodiments described herein. The GUI 100 of FIG. 2 is the same as the GUI 100 of FIG. 1, except that the GUI 100 of FIG. 2 displays a circle 214. In some embodiments, such as that of FIG. 2, the center of the circle 214 may be at the center of the sample volume 104 and the diameter of the circle 214 may be the length of the vessel line 106. After the circle 214 is displayed, upon detecting a dragging movement (e.g., by a finger or a mouse cursor) from the caliper 112, the processing device may cause the vessel line 106 to rotate about the sample volume 104. In some embodiments, if the dragging movement covers a distance x in the horizontal direction and a distance y in the vertical direction, then the processing device may cause the vessel line 106 to rotate about the sample volume 104 by θ=tan⁻¹ y/x degrees. The circle 214 may therefore highlight the possible orientations of the vessel line 106, given a location of the sample volume 104. When the dragging movement ceases, the circle 214 may disappear. Because a user may modify the direction of the vessel line 106 by touching the caliper 112 and not the vessel line 106 itself, the vessel line 106 may not be obscured as its direction changes, which may help the user to more easily select the direction of the vessel line 106.

In some embodiments, the processing device may change the location of the sample volume 104 upon detecting a dragging movement that begins at any location in the GUI 100 within a circle, where the center of the circle 214 may be at the center of the sample volume 104 and the diameter of the circle 214 may be the length of the vessel line 106. In other words, the circle, though not displayed, may be the same as the circle 214. The processing may also change the location of the sample volume 104 upon detecting a dragging movement that begins at any location within the circle 214 when the circle 214 is displayed. The processing device may then cause the location of the sample volume 104 to change based on the dragging movement. In other words, if the dragging movement proceeds from the location within the circle a certain distance in the horizontal direction and a certain distance in the vertical direction, then the processing device may change the location of the sample volume 104 by the distance in the horizontal direction and the distance in the vertical direction.

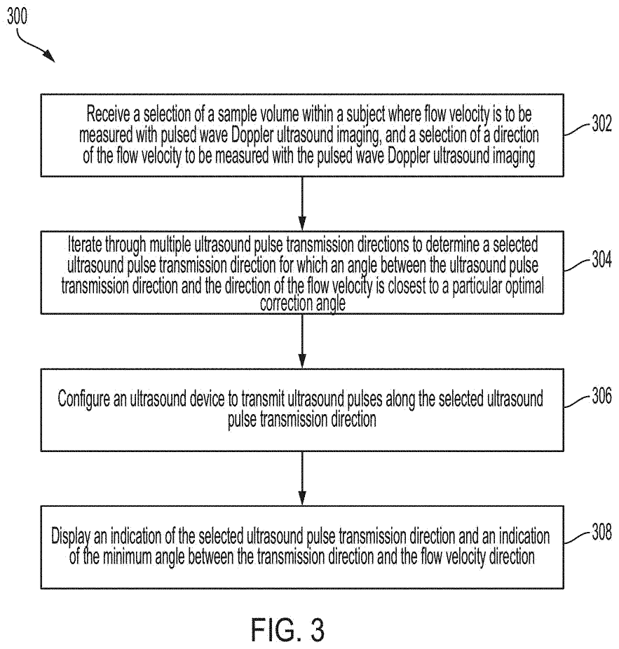

As described with reference to FIG. 3, the processing device may automatically calculate the direction of the beam line 108 based on the location of the sample volume and the direction of the vessel line 106. After the processing device determines the direction of the beam line 108, the processing device may display the beam line 108 and display the correction angle, namely the angle between the beam line and vessel line, in the angle indicator 110. In some embodiments, it may not be possible for the beam line 108 to be modified directly by the user, but rather may be modified by the user modifying the location of the sample volume 104 and/or the direction of the vessel line 106.

In some embodiments, there may be multiple (e.g., two) different options for the size of the sample volume 104, and therefore multiple different options for the size of the sample volume within the subject. In some embodiments, if the correction angle is larger than a threshold angle (e.g., 90 degrees), then some aspect (e.g., color) of the angle indicator 110 may change to indicate that the location of the sample volume 104 and/or the direction of the vessel line 106 are suboptimal.

FIG. 3 illustrates a flow chart of an example process 300 for pulsed wave Doppler ultrasound imaging, in accordance with certain embodiments described herein. The process 300 may be performed using a processing device in operative communication with an ultrasound device. For example, the processing device may be a mobile phone, tablet, or laptop. The ultrasound device and the processing device may communicate over a wired communication link (e.g., over Ethernet, a Universal Serial Bus (USB) cable or a Lightning cable) or over a wireless communication link (e.g., over a BLUETOOTH, WiFi, or ZIGBEE wireless communication link). The process 300 generally includes using the processing device to automatically calculate an ultrasound pulse transmission direction.

In act 302, the processing device receives a selection of a sample volume within a subject where flow velocity is to be measured with pulsed wave Doppler ultrasound imaging, and a selection of a direction of the flow velocity to be measured with the pulsed wave Doppler ultrasound imaging. In some embodiments, the processing device may receive the selection of the sample volume and the direction of the flow velocity through a graphical user interface (e.g., the GUI 100) that depicts an ultrasound image (e.g., the ultrasound image 102) and is displayed on a display screen of the processing device. The GUI may be touch activated (e.g., may be displayed on a touch-sensitive display screen). In some embodiments, the processing device may receive the selection of the sample volume using the sample volume 104 on the GUI 100. Further description of the sample volume 104 may be found with reference to FIGS. 1-2. In some embodiments, the processing device may receive the selection of the direction of the flow velocity using the vessel line 106 on the GUI 100. Further description of the vessel line 106 may be found with reference to FIGS. 1-2. The process 300 proceeds from act 302 to act 304.

In act 304, the processing device iterates through multiple ultrasound pulse transmission directions to determine a selected ultrasound pulse transmission direction for which an angle between the ultrasound pulse transmission direction and the direction of the flow velocity is closest to a particular optimal correction angle. In some embodiments, the optimal correction angle may be zero. In some embodiments, the optimal correction angle may be non-zero. In some embodiments, the optimal correction angle particular angle may be predetermined and stored in memory of the processing device, and the processing device may retrieve that stored angle. In some embodiments, the stored optimal correction angle may be specific to the particular anatomical region or structure being imaged. For example, the optimal correction angle stored for cardiac imaging may be zero, while the optimal correction angle for carotid and vascular access imaging may be non-zero. Thus, the user may select an option from the processing device to image a particular type of anatomy, and the processing device may retrieve the stored optimal correction angle specific to that anatomy. In some embodiments, the processing device may provide the user with an option to input the optimal correction angle, and the processing device may use that inputted angle.

In some embodiments, the processing device may iterate through the multiple ultrasound pulse transmission directions by iterating through multiple starting positions for transmission of the ultrasound pulses from the transducer array of the ultrasound device. In some embodiments, the processing device may iterate through multiple starting positions for transmission of the ultrasound pulses from the transducer array of the ultrasound device by iterating through multiple subsets of ultrasound transducers in the transducer array from which to transmit the ultrasound pulses. In some embodiments, the processing device may uniquely determine an ultrasound pulse transmission direction for a given starting position and a given sample volume. In such embodiments, for each different starting position for transmission of the ultrasound pulses from the transducer array of the ultrasound device, the processing device may determine what the angle would be between the ultrasound pulses directed from the starting position to the sample volume and the direction of the flow velocity to be measured. The processing device may select the starting position for transmission of the ultrasound pulses that results in the angle that is closest to the optimal correction angle (e.g., zero or a non-zero angle, and which may be selected as described above). The direction from the selected starting position to the sample volume may therefore be the selected ultrasound pulse transmission direction. This direction may be considered the best or optimal transmission direction.

In some embodiments, the processing device may iterate through different ultrasound pulse transmission directions from a given starting position. In such embodiments, for each different starting position and each different transmission direction from that starting position, the processing device may determine what the angle would be between the ultrasound pulses directed from the starting position to the sample volume and the direction of the flow velocity to be measured. The processing device may select the starting position and the transmission direction from that starting positions that results in the angle that is closest to a particular optimal correction angle. The selected direction from the selected starting position to the sample volume may therefore be the selected ultrasound pulse transmission direction. This direction may be considered the best or optimal transmission direction. In some embodiments, rather than iterating over multiple starting positions in order to iterate over multiple transmission directions, the processing device may iterate over a different parameter that affects the transmission direction.

In some embodiments, there may be a limited number (e.g., three) of options for ultrasound pulse transmission directions through which to iterate, while in other embodiments there may be more options through which to iterate. The number of ultrasound pulse transmission directions through which to iterate may be the same as the number of starting positions for ultrasound pulse transmission through which to iterate.

In some embodiments, there may be a limit to the ultrasound pulse transmission direction that the processing device may use. This limit may be specific to the anatomical region or structure being imaged, and may help to avoid steering the ultrasound pulse too steeply out of the transducer array. Thus, if the user selects an option from the processing device to image a particular anatomical region or structure, then the processing device may impose a limit angle on the ultrasound pulse transmission direction, where the limit angle is specific to that anatomical region or structure. In some embodiments, the limit angle for the ultrasound pulse transmission direction for carotid and vascular access imaging may be a smaller angle than the limit angle for cardiac imaging. The processing device may determine whether the best transmission direction calculated as described above is a larger angle than the limit angle. If the calculated best transmission direction is a larger angle than the limit angle, then the processing device may select another transmission direction. For example, the processing device may select the next-best transmission direction. This next-best transmission direction may be determined using the same iteration processes described above. In other words, the next-best transmission direction may be the ultrasound pulse transmission direction for which an angle between the ultrasound pulse transmission direction and the direction of the flow velocity is next closest to the particular optimal correction angle after the transmission direction found to violate the limit angle. The process 300 proceeds from act 304 to act 306.

In act 306, the processing device configures the ultrasound device to transmit ultrasound pulses along the selected ultrasound pulse transmission direction. In some embodiments, the processing device may transmit commands to the ultrasound device to configure the ultrasound device to transmit ultrasound pulses along the selected ultrasound pulse transmission direction. In some embodiments, the processing device may automatically (i.e., without further user input) configure the ultrasound device to transit the ultrasound pulses along the selected ultrasound pulse transmission direction. The process 300 proceeds from act 306 to act 308.

In act 308, the processing device displays an indication of the selected ultrasound pulse transmission direction and an indication of the angle, which may be a minimum angle, between the ultrasound pulse transmission direction and the flow velocity direction. In some embodiments, the indication of the selected ultrasound pulse transmission direction and the indication of the angle may be displayed on a GUI (e.g., the GUI 100) that depicts an ultrasound image (e.g., the ultrasound image 102) and is displayed on a display screen of the processing device. The indication of the selected ultrasound pulse transmission direction may include a line extending from the top of the ultrasound image, through the sample volume, and to the bottom of the ultrasound image. The path of this line in the ultrasound image may correspond to the direction in which the ultrasound device transmits ultrasound pulses through the subject. In some embodiments, act 308 may occur before, or simultaneously with, act 306. In some embodiments, act 308 may be omitted.

In some embodiments, the indication of the angle between the ultrasound pulse transmission direction and the flow velocity direction may be the angle indicator 110. In some embodiments, if this angle is larger than a threshold angle (e.g., 90 degrees), then some aspect (e.g., color) of the indication of the angle may change to indicate that the location of the sample volume and/or the direction of the flow velocity are suboptimal.

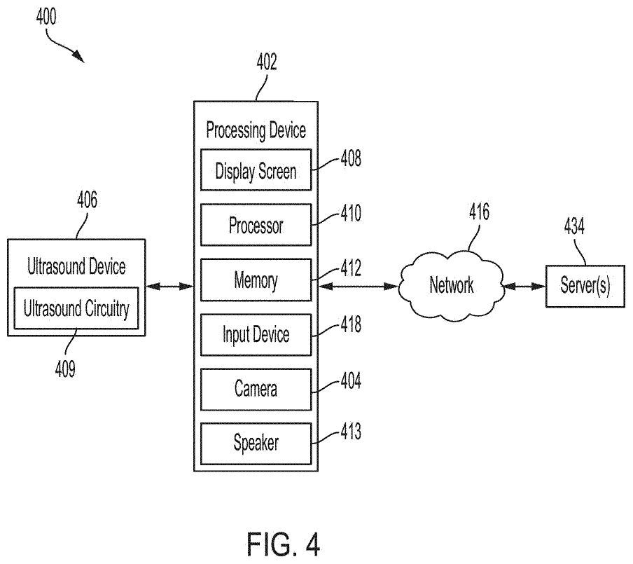

FIG. 4 illustrates a schematic block diagram of an example ultrasound system 400 upon which various aspects of the technology described herein may be practiced. The ultrasound system 400 includes an ultrasound device 406, a processing device 402, a network 416, and one or more servers 434.

The ultrasound device 406 includes ultrasound circuitry 409. The processing device 402 includes a camera 404, a display screen 408, a processor 410, a memory 412, an input device 418, and a speaker 413. The processing device 402 is in wired (e.g., through a lightning connector or a mini-USB connector) and/or wireless communication (e.g., using BLUETOOTH, ZIGBEE, and/or WiFi wireless protocols) with the ultrasound device 406. The processing device 402 is in wireless communication with the one or more servers 434 over the network 416. However, the wireless communication with the processing device 434 is optional.

The ultrasound device 406 may be configured to generate ultrasound data that may be employed to generate an ultrasound image. The ultrasound device 406 may be constructed in any of a variety of ways. In some embodiments, the ultrasound device 406 includes a transmitter that transmits a signal to a transmit beamformer which in turn drives transducer elements within a transducer array to emit pulsed ultrasonic signals into a structure, such as a patient. The pulsed ultrasonic signals may be back-scattered from structures in the body, such as blood cells or muscular tissue, to produce echoes that return to the transducer elements. These echoes may then be converted into electrical signals by the transducer elements and the electrical signals are received by a receiver. The electrical signals representing the received echoes are sent to a receive beamformer that outputs ultrasound data. The ultrasound circuitry 409 may be configured to generate the ultrasound data. The ultrasound circuitry 409 may include one or more ultrasonic transducers monolithically integrated onto a single semiconductor die. The ultrasonic transducers may include, for example, one or more capacitive micromachined ultrasonic transducers (CMUTs), one or more CMOS (complementary metal-oxide-semiconductor) ultrasonic transducers (CUTs), one or more piezoelectric micromachined ultrasonic transducers (PMUTs), and/or one or more other suitable ultrasonic transducer cells. In some embodiments, the ultrasonic transducers may be formed the same chip as other electronic components in the ultrasound circuitry 409 (e.g., transmit circuitry, receive circuitry, control circuitry, power management circuitry, and processing circuitry) to form a monolithic ultrasound device. The ultrasound device 406 may transmit ultrasound data and/or ultrasound images to the processing device 402 over a wired (e.g., through a lightning connector or a mini-USB connector) and/or wireless (e.g., using BLUETOOTH, ZIGBEE, and/or WiFi wireless protocols) communication link.

Referring now to the processing device 402, the processor 410 may include specially-programmed and/or special-purpose hardware such as an application-specific integrated circuit (ASIC). For example, the processor 410 may include one or more graphics processing units (GPUs) and/or one or more tensor processing units (TPUs). TPUs may be ASICs specifically designed for machine learning (e.g., deep learning). The TPUs may be employed to, for example, accelerate the inference phase of a neural network. The processing device 402 may be configured to process the ultrasound data received from the ultrasound device 406 to generate ultrasound images for display on the display screen 408. The processing may be performed by, for example, the processor 410. The processor 410 may also be adapted to control the acquisition of ultrasound data with the ultrasound device 406. The ultrasound data may be processed in real-time during a scanning session as the echo signals are received. In some embodiments, the displayed ultrasound image may be updated a rate of at least 5 Hz, at least 10 Hz, at least 20 Hz, at a rate between 5 and 60 Hz, at a rate of more than 20 Hz. For example, ultrasound data may be acquired even as images are being generated based on previously acquired data and while a live ultrasound image is being displayed. As additional ultrasound data is acquired, additional frames or images generated from more-recently acquired ultrasound data are sequentially displayed. Additionally, or alternatively, the ultrasound data may be stored temporarily in a buffer during a scanning session and processed in less than real-time.

The processing device 402 may be configured to perform certain of the processes (e.g., the process 300) described herein using the processor 410 (e.g., one or more computer hardware processors) and one or more articles of manufacture that include non-transitory computer-readable storage media such as the memory 412. The processor 410 may control writing data to and reading data from the memory 412 in any suitable manner. To perform certain of the processes described herein, the processor 410 may execute one or more processor-executable instructions stored in one or more non-transitory computer-readable storage media (e.g., the memory 412), which may serve as non-transitory computer-readable storage media storing processor-executable instructions for execution by the processor 410. The camera 404 may be configured to detect light (e.g., visible light) to form an image. The camera 404 may be on the same face of the processing device 402 as the display screen 408. The display screen 408 may be configured to display images and/or videos, and may be, for example, a liquid crystal display (LCD), a plasma display, and/or an organic light emitting diode (OLED) display on the processing device 402. The input device 418 may include one or more devices capable of receiving input from a user and transmitting the input to the processor 410. For example, the input device 418 may include a keyboard, a mouse, a microphone, touch-enabled sensors on the display screen 408, and/or a microphone. The display screen 408, the input device 418, the camera 404, and the speaker 409 may be communicatively coupled to the processor 410 and/or under the control of the processor 410.

It should be appreciated that the processing device 402 may be implemented in any of a variety of ways. For example, the processing device 402 may be implemented as a handheld device such as a mobile smartphone or a tablet. Thereby, a user of the ultrasound device 406 may be able to operate the ultrasound device 406 with one hand and hold the processing device 402 with another hand. In other examples, the processing device 402 may be implemented as a portable device that is not a handheld device, such as a laptop. In yet other examples, the processing device 402 may be implemented as a stationary device such as a desktop computer. The processing device 402 may be connected to the network 416 over a wired connection (e.g., via an Ethernet cable) and/or a wireless connection (e.g., over a WiFi network). The processing device 402 may thereby communicate with (e.g., transmit data to) the one or more servers 434 over the network 416. For further description of ultrasound devices and systems, see U.S. patent application Ser. No. 15/415,434 titled “UNIVERSAL ULTRASOUND DEVICE AND RELATED APPARATUS AND METHODS,” filed on Jan. 25, 2017 and published as U.S. Pat. App. Publication No. 2017-0360397 A1 (and assigned to the assignee of the instant application).

FIG. 4 should be understood to be non-limiting. For example, the ultrasound system 400 may include fewer or more components than shown and the processing device 402 and ultrasound device 406 may include fewer or more components than shown. In some embodiments, the processing device 402 may be part of the ultrasound device 406.

Various aspects of the present disclosure may be used alone, in combination, or in a variety of arrangements not specifically discussed in the embodiments described in the foregoing and is therefore not limited in its application to the details and arrangement of components set forth in the foregoing description or illustrated in the drawings. For example, aspects described in one embodiment may be combined in any manner with aspects described in other embodiments.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The terms “approximately” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Having described above several aspects of at least one embodiment, it is to be appreciated various alterations, modifications, and improvements will readily occur to those skilled in the art. Such alterations, modifications, and improvements are intended to be object of this disclosure. Accordingly, the foregoing description and drawings are by way of example only.

What is claimed is:

1. An apparatus, comprising:
a processing device in operative communication with an ultrasound device, the processing device configured to automatically calculate an ultrasound pulse transmission direction for pulsed wave Doppler ultrasound imaging.

2. The apparatus of claim 1, wherein the processing device is further configured to configure the ultrasound device to transmit ultrasound pulses along the ultrasound pulse transmission direction.

3. The apparatus of claim 1, wherein the processing device is configured to automatically calculate the ultrasound pulse transmission direction based on:
a selected sample volume within a subject where flow velocity is to be measured with the pulsed wave Doppler ultrasound imaging; and
a selected direction of the flow velocity to be measured with the pulsed wave Doppler ultrasound imaging.

4. The apparatus of claim 1, wherein the processing device is configured, when automatically calculating the ultrasound pulse transmission direction for the pulsed wave Doppler ultrasound imaging, to:
receive a selection of a sample volume within a subject where flow velocity is to be measured with the pulsed wave Doppler ultrasound imaging, and a selection of a direction of the flow velocity to be measured with the pulsed wave Doppler ultrasound imaging; and
iterate through multiple ultrasound pulse transmission directions to determine a selected ultrasound pulse transmission direction for which an angle between the ultrasound pulse transmission direction and the direction of the flow velocity is closest to a particular optimal correction angle.

5. The apparatus of claim 4, wherein the processing device is further configured to display an indication of the selected ultrasound pulse transmission direction and an indication of the angle between the ultrasound pulse transmission direction and the direction of the flow velocity.

6. The apparatus of claim 5, wherein the processing device is configured, when displaying the indication of the selected ultrasound pulse transmission direction, to:
display, on a graphical user interface that depicts an ultrasound image collected by the ultrasound device, a line extending from a top of the ultrasound image, through an indication of the sample volume, and to a bottom of the ultrasound image, such that a path of the line in the ultrasound image corresponds to the selected ultrasound pulse transmission direction.

7. The apparatus of claim 4, wherein the processing device is configured, when iterating through the multiple ultrasound pulse transmission directions, to iterate through multiple starting positions for transmission of the ultrasound pulses from the ultrasound device.

8. The apparatus of claim 7, wherein the processing device is configured, when iterating through the multiple starting positions for transmission of the ultrasound pulses from the ultrasound device, to iterate through multiple subsets of ultrasound transducers in a transducer array of the ultrasound device from which to transmit the ultrasound pulses.

9. The apparatus of claim 7, wherein the processing device is configured, when iterating through the multiple starting positions for transmission of the ultrasound pulses from the ultrasound device, to:
for each respective starting position of the multiple starting positions for transmission of the ultrasound pulses from the ultrasound device, determine an angle that would be between the ultrasound pulses directed from the respective starting position to the sample volume and the direction of the flow velocity to be measured; and
select a starting position for transmission of the ultrasound pulses such that the angle is closest to the particular optimal correction angle.

10. The apparatus of claim 4, wherein the particular optimal correction angle is zero.

11. The apparatus of claim 4, wherein the processing device is configured to use a zero angle for the particular optimal correction angle for cardiac imaging.

12. The apparatus of claim 4, wherein the processing device is configured to use a nonzero angle for the particular optimal correction angle for carotid and/or vascular access imaging.

13. The apparatus of claim 4, wherein the processing device is configured to use a zero angle for the particular optimal correction angle for cardiac imaging and a nonzero angle for the particular optimal correction angle for carotid and/or vascular access imaging.

14. The apparatus of claim 4, wherein the processing device is configured to provide a user with an option to input the particular optimal correction angle.

15. The apparatus of claim 4, wherein the processing device is configured, when automatically calculating the ultrasound pulse transmission direction, to determine whether the calculated ultrasound pulse transmission direction is larger than a particular limit angle.

16. The apparatus of claim 15, wherein the processing device is configured, if the calculated ultrasound pulse transmission direction is larger than the particular limit angle, to select a second ultrasound pulse transmission direction.

17. The apparatus of claim 16, wherein an angle between the second ultrasound pulse transmission direction and the direction of the flow velocity is next closest to the particular optimal correction angle after the ultrasound pulse transmission direction determined to be larger than the particular limit angle.

18. The apparatus of claim 15, wherein the processing device is configured to use a first angle for the particular limit angle for carotid and/or vascular access imaging that is smaller than a second angle used for the limit angle for cardiac imaging.

19. A method, comprising:
automatically calculating, using a processing device in operative communication with an ultrasound device, an ultrasound pulse transmission direction for pulsed wave Doppler ultrasound imaging.

20. The method of claim 19, wherein automatically calculating the ultrasound pulse transmission direction for the pulsed wave Doppler ultrasound imaging comprises:
receiving a selection of a sample volume within a subject where flow velocity is to be measured with the pulsed wave Doppler ultrasound imaging, and a selection of a direction of the flow velocity to be measured with the pulsed wave Doppler ultrasound imaging; and
iterating through multiple ultrasound pulse transmission directions to determine a selected ultrasound pulse transmission direction for which an angle between the ultrasound pulse transmission direction and the direction of the flow velocity is closest to a particular optimal correction angle.
